(19) United States
(12) Patent Application Publication
(10) Pub. No.: US 2022/0259265 A1
(43) Pub. Date: Aug. 18, 2022

(54) Inhibitors and Use Thereof in Cancer Treatment

(71) Applicant: The University of Sydney, Sydney, New South Wales (AU)

(72) Inventor: Robert Baxter, St Leonards, New South Wales (AU)

(21) Appl. No.: 17/628,061

(22) PCT Filed: Jul. 30, 2020
(86) PCT No.: PCT/AU2020/050779

§ 371 Date: Jan. 18, 2022

(30) Foreign Application Priority Data
Jul. 30, 2019 (AU) 2019902712

(51) Int. Cl. C07K 7/08; A61K 31/7048; A61K 31/502; A61K 31/4184; A61P 35/00

(52) U.S. Cl. CPC C07K 7/08; A61K 31/7048; A61K 38/00; A61K 31/4184; A61P 35/00; A61K 31/502

(57) ABSTRACT

The invention generally relates to inhibitors of DNA double strand break (DSB) repair in cancer cells exposed to DNA-damaging chemotherapy drugs or radiotherapy. In particular, agents that inhibit binding between insulin-like growth factor binding protein-3 (IGFBP-3) and non-POU (pituitary-specific Pit-1, octamer-binding proteins Oct-1 and Oct-2, and neural Unc-86) domain-containing octamer-binding protein (NONO) and methods of using such agents to enhance chemosensitivity or radiosensitivity in cancer treatment are disclosed.

### FIELD OF THE INVENTION

The present invention generally relates to inhibitors of DNA double-strand break (DSB) repair. In particular, the present invention relates to agents that inhibit binding between insulin-like growth factor binding protein-3 (IGFBP-3) and non-POU (pituitary-specific Pit-1, octamer-binding proteins Oct-1 and Oct-2, and neural Unc-86) domain-containing octamer-binding protein (NONO), and methods of using such agents to enhance chemosensitivity or radiosensitivity in cancer treatment.

### BACKGROUND

Any discussion of the prior art throughout the specification should in no way be considered as an admission that such prior art is widely known or forms part of common general knowledge in the field.

The mechanism of action of many chemo- and radiotherapies is the induction of DSB in cancer cell DNA. In response, cancer cells can either enter a program of cell death or DSB repair. A common DSB repair mechanism is non-homologous end-joining (NHEJ). This pathway is referred to as “non-homologous” because unlike the other classic DSB repair mechanism, homologous recombination (HR), NHEJ does not require a homologous template for repair of the DNA lesion. As DNA damage repair makes chemo- and radiotherapy less effective, agents that inhibit DNA repair pathways enhance the specificity and effectiveness of chemo- and radiotherapy and may help overcome cancer treatment resistance.

Triple Negative Breast Cancers (TNBC) are unresponsive to estrogen receptor or human epidermal growth factor receptor 2 (HER2) directed treatments. TNBC is a more aggressive form of breast cancer with a high prevalence in younger women and is associated with an unfavorable prognosis. There has been limited therapeutic progress for treating TNBC in the past several decades and cytotoxic chemotherapy is still the standard of care. However, their responsiveness may be blunted by DNA DSB repair. There is thus an urgent unmet need to develop effective agents for sensitizing DNA-damaging chemotherapy drugs or radiotherapy, especially for this patient population.

It is an object of the present invention to overcome or ameliorate at least one of the disadvantages of the prior art, or to provide a useful alternative.

### SUMMARY OF THE INVENTION

Chemotherapies and radiotherapies induce DNA DSB in cancer cell DNA. Cancer cells in turn can either enter a program of cell death or DNA damage repair. An important pathway for DNA DSB repair is NHEJ. The protein IGFBP-3 is involved in DSB repair by NHEJ and the inventor has unexpectedly found that DNA- and RNA-binding protein NONO (and its dimerization partner splicing factor, proline/glutamine-rich (SFPQ)) interacts with IGFBP-3 in TNBC cell lines exposed to chemotherapy drugs and promotes DNA DSB repair.

The invention generally relates to inhibitors of DNA DSB repair in cancer cells exposed to DNA-damaging chemotherapy drugs or radiotherapy. In particular, the present invention relates to agents that inhibit binding between IGFBP-3 and NONO, and methods of using such agents to enhance chemosensitivity or radiosensitivity in cancer treatment.

Provided are agents that inhibit the interaction between IGFBP-3 and NONO and inhibit DNA DSB repair. Such agents enhance chemosensitivity or radiosensitivity. The agents as described herein may be a small molecule, substance or compound that inhibits the interaction between IGFBP-3 and NONO and thus inhibits DNA DSB repair following chemotherapy or radiotherapy.

Provided are isolated peptides that inhibit the interaction between NONO and IGFBP-3. The peptides of the invention may be derived from the full length sequence of mature human IGFBP-3.

Provided are methods for enhancing chemosensitivity or radiosensitivity in cancer treatment comprising administering to a subject in need thereof an agent that inhibits the interaction between NONO and IGFBP-3. In particular, provided are methods for enhancing chemosensitivity or radiosensitivity in TNBC treatment. Agents that inhibit the interaction between NONO and IGFBP-3 may be suitable for neoadjuvant or adjuvant therapy to be used in conjunction with radiotherapies or other chemotherapeutic approaches.

Provided is a therapy for enhancing chemosensitivity or radiosensitivity in cancer treatment comprising administering an agent that inhibits the interaction between NONO and IGFBP-3. Exemplary cancers that may be treated include, but are not limited to, breast cancer, prostate cancer, pancreatic cancer, glioblastoma and the like. In particular, provided is a therapy for enhancing chemosensitivity or radiosensitivity in TNBC treatment.

In a first aspect, the invention provides an agent that inhibits the interaction between IGFBP-3 and NONO.

In a second aspect, the invention provides an isolated peptide comprising residues:

X₁-X₂-X₃-X₄-X₅-X₆-X₇-X₈-X₉,

wherein X₁ is His, X₂ is Leu, X₃ is Lys, X₄ is Phe, X₅ is Leu, X₆ is Asn, X₇ is Val, X₈ is Leu and X₉ is Ser, or conservative substitutions thereof,

or a pharmaceutically acceptable salt of the peptide.

In certain embodiments, the peptide comprises the sequence:

His-Leu-Lys-Phe-Leu-Asn-Val-Leu-Ser-Pro-Arg-Gly,

or conservative substitutions thereof,

or a pharmaceutically acceptable salt of the peptide.

In some embodiments, the peptide comprises the sequence:

Thr-Leu-Asn-His-Leu-Lys-Phe-Leu-Asn-Val-Leu-Ser,

or conservative substitutions thereof,

or a pharmaceutically acceptable salt of the peptide.

In a third aspect, the invention provides an isolated peptide comprising residues:

X₁-X₂-X₃-X₄-X₅-X₆-X₇-X₈-X₉,

wherein X₁ is His, X₂ is Leu, X₃ is Lys, X₄ is Phe, X₅ is Leu, X₆ is Asn, X₇ is Val, X₈ is Leu and X₉ is Ser, or conservative substitutions thereof,
or a pharmaceutically acceptable salt of the peptide, wherein the peptide inhibits the interaction between IGFBP-3 and NONO.

In certain embodiments, the peptide comprises the sequence:

His-Leu-Lys-Phe-Leu-Asn-Val-Leu-Ser-Pro-Arg-Gly,

or conservative substitutions thereof,
or a pharmaceutically acceptable salt of the peptide, wherein the peptide inhibits the interaction between IGFBP-3 and NONO.

In some embodiments, the peptide comprises the sequence:

Thr-Leu-Asn-His-Leu-Lys-Phe-Leu-Asn-Val-Leu-Ser,

or conservative substitutions thereof,
or a pharmaceutically acceptable salt of the peptide, wherein the peptide inhibits the interaction between IGFBP-3 and NONO.

In a fourth aspect, the invention provides a pharmaceutical composition comprising an agent of the invention, or an isolated peptide of the invention and optionally at least one pharmaceutically acceptable excipient. In some embodiments, the pharmaceutical composition further comprises a chemotherapeutic agent, a radiomimetic agent or a PARP inhibitor. In a related embodiment, the chemotherapeutic agent is selected from the group consisting of a bifunctional alkylator, a monofunctional alkylator, a topoisomerase inhibitor, an antimetabolite, a replication inhibitor and a platinum drug. In some embodiments, the chemotherapeutic agent is etoposide. In certain embodiments, the PARP inhibitor is veliparib. In some embodiments, the PARP inhibitor is olaparib.

In a fifth aspect, the invention provides a method of enhancing chemosensitivity or radiosensitivity in cancer treatment comprising administering to a subject in need thereof a therapeutically effective amount of an agent of the invention, an isolated peptide of the invention or a pharmaceutical composition of the invention, wherein the cancer is an IGFBP-3 expressing cancer. In some embodiments, the IGFBP-3 expressing cancer is breast cancer, prostate cancer, pancreatic cancer or glioblastoma cancer. In certain embodiments, the IGFBP-3 expressing cancer is Triple Negative Breast Cancer (TNBC).

In a sixth aspect, the invention provides a method of enhancing chemosensitivity or radiosensitivity in TNBC treatment comprising administering to a subject in need thereof a therapeutically effective amount of an agent of the invention, an isolated peptide of the invention or a pharmaceutical composition of the invention.

In a seventh aspect, the invention provides use of an agent of the invention, or an isolated peptide of the invention in the manufacture of a medicament for enhancing chemosensitivity or radiosensitivity in cancer treatment, wherein the cancer is an IGFBP-3 expressing cancer.

In an eighth aspect, the invention provides use of an agent of the invention, or an isolated peptide of the invention in the manufacture of a medicament for enhancing chemosensitivity or radiosensitivity in TNBC treatment.

In some embodiments, the invention provides an agent of the invention for use in a method of enhancing chemosensitivity or radiosensitivity in cancer treatment, wherein the cancer is an IGFBP-3 expressing cancer. In a related embodiment, the IGFBP-3 expressing cancer is breast cancer, prostate cancer, pancreatic cancer or glioblastoma cancer

In some embodiments, the invention provides an agent of the invention for use in a method of enhancing chemosensitivity or radiosensitivity in TNBC treatment.

In certain embodiments, the invention provides an isolated peptide of the invention or a pharmaceutically acceptable salt thereof for use in a method of enhancing chemosensitivity or radiosensitivity in cancer treatment, wherein the cancer is an IGFBP-3 expressing cancer. In a related embodiment, the IGFBP-3 expressing cancer is breast cancer, prostate cancer, pancreatic cancer or glioblastoma cancer.

In some embodiments, the invention provides an isolated peptide of the invention or a pharmaceutically acceptable salt thereof for use in a method of enhancing chemosensitivity or radiosensitivity in TNBC treatment.

Methods of synthesizing or generating an agent or a peptide herein disclosed are not particularly limited and any suitable method may be used.

Definitions

Unless the context clearly requires otherwise, throughout the description and the claims, the words “comprise”, “comprising” and the like are to be construed in an inclusive sense as opposed to an exclusive or exhaustive sense; that is to say, in the sense of “including, but not limited to”.

As used herein, the singular forms “a,” “an” and “the” refer to “one or more” when used in this application. Thus, for example, reference to “a sample” includes a plurality of such samples, and so forth.

As used herein, the term “about” can mean within 1 or more standard deviation per the practice of the art. Alternatively, “about” can mean a range of up to 20%. When particular values are provided in the specification and claims the meaning of “about” should be assumed to be within an acceptable error range for that particular value.

The term “agent” refers to a molecule or a substance. An agent as described herein “inhibits” the interaction between IGFBP-3 and NONO. The term “inhibits” in this context thus refers to slowing down or preventing the interaction. For example, an agent that inhibits the interaction between IGFBP-3 and NONO as described herein slows down or prevents IGFBP-3 binding to NONO, and in this way diminishes or prevents DNA DSB repair mechanism, preferably by NHEJ.

The term “chemosensitivity” and “radiosensitivity” as referred to herein is the relative susceptibility of cells, tissues, organs or organisms to the effect of chemotherapeutic agents and ionizing radiation, respectively.

The term “peptide” as used herein includes but is not limited to, two or more amino acids, or residues covalently linked by a peptide bond or equivalent. In certain embodiments, amino acids may be linked by non-natural and non-peptide bonds. In the context of the present invention, it is to be understood that the term “isolated” as used herein i.e. “an isolated peptide” is intended to refer to a peptide that is separated from the natural environment, e.g. the human body. The term “isolated peptide” as used herein includes peptides based on the complete full-length human IGFBP-3 sequence but are not part of the full protein, i.e. they are isolated from it. In other words, isolated peptides provided herein do not necessarily comprise the complete full-length human IGFBP-3 sequence. However, the present invention does not intend to exclude embodiments wherein the isolated peptide is a portion of a larger peptide, such as a pre-pro-protein or a polypeptide that comprises an amino acid sequence that can be processed (e.g. by cleavage) into a number of smaller peptides following expression. Isolated peptides described herein include but are not limited to chemically synthesized peptides, recombinant peptides, and peptides that have been modified. The person skilled in the art will appreciate that a number of modifications can be made to the peptides to improve peptide stability and pharmacokinetic properties, for example, peptide absorption, distribution, metabolism, and excretion (ADME) properties. The peptides as herein described may be modified to form a cyclic structure (i.e. a cyclic peptide). Methods of modifying a peptide as herein disclosed to a cyclic structure are not particularly limited and any suitable methods may be used. The peptides as herein described may be modified such that the peptide includes non-peptide bonds or other synthetic modifications such as the use of non-natural amino acids. These modifications may render the peptides more stable while in the body or more capable of penetrating into cells.

The term “interaction” as used herein refers to either a direct or indirect interaction. In the context of the present invention, an agent that inhibits the “interaction” between IGFBP-3 and NONO therefore refers to, but is not limited to, either inhibiting the physical binding of the two proteins (direct interaction) or modulating the expression of one or both of the proteins (indirect interaction). The term “interaction” as used herein may also be taken to mean that the proteins exist as part of the same multi-protein complex, independent of whether the proteins are in direct physical contact. Protein interactions can be determined by various methods including but is not limited to the yeast two-hybrid system, affinity chromatography, co-immunoprecipitation, proximity ligation assay, subcellular fractionation and isolation of large molecular complexes. Each of these methods is well characterised and can be readily performed by one skilled in the art.

The term “conservative substitutions” used herein refers to replacing one amino acid with another having similar structural and/or chemical properties, such as the replacement of a leucine with an isoleucine or valine, an aspartate with a glutamate, or a threonine with a serine. A “conservative substitution” of a particular sequence refers to substitution of those amino acids that are not critical for peptide activity or substitution of amino acids with other amino acids having similar properties, for example acidic, basic, positively or negatively charged, polar or non-polar etc, such that the substitution of even critical amino acids does not reduce the activity of the peptide (i.e. the ability of the peptide to inhibit NONO-IGFBP-3 interaction). Conservative substitutions of functionally similar amino acids are well known in the art. For example, the following six groups each contain amino acids that are conservative substitutions for one another: i) Alanine (A), Serine (S), Threonine (T); ii) Aspartic acid (D), Glutamic acid (E); iii) Asparagine (N), Glutamine (Q); iv) Arginine (R), Lysine (K); v) Isoleucine (I), Leucine (L), Methionine (M), Valine (V); and vi) Phenylalanine (F), Tyrosine (Y), Tryptophan (W). In some embodiments, individual substitutions of a single amino acid or a small percentage of amino acids can also be considered “conservative substitutions” if the substitution does not reduce the activity of the peptide. The choice of conservative amino acids may be selected based on the location of the amino acid to be substituted in the peptide, for example if the amino acid is on the exterior of the peptide and expose to solvents, or on the interior and not exposed to solvents.

The three-letter abbreviations or one-letter abbreviations of amino acids are known and standard in the art, and include for example alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (Ile or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V). Non-traditional and non-natural amino acids are also within the scope of the invention. The amino acids described herein may be in the “L” or “D” stereoisomeric form. In the absence of a “D” or “L” designation, an amino acid in the three-letter abbreviation is in the “L” form.

Radiotherapy intended in the present invention is commonly used in this technical field and can be performed according to protocols known to those skilled in the art. For example, radiotherapy as used herein includes but is not limited to irradiation with cesium, iridium, iodine, cobalt or other suitable isotopes. Radiotherapy may be systemic irradiation or local irradiation. The dose fractionation and duration of the radiotherapy intended in the present invention are not particularly limiting. Exemplary methods include radiotherapy divided into 25 to 30 fractions, over about 5 to 6 weeks, and performed for 2 to 3 minutes per day.

As used herein, the term “radiomimetic agent” refers to cytotoxic agents that damage DNA in such a way that the lesions produced in DNA are similar to those resulting from ionising radiation. Examples of radiomimetic agents which cause DNA strand breaks include but is not limited to bleomycin, doxorubicin (adriamycin), 5-fluorouracil (5 FU), neocarzinostatin, alkylating agents and other agents that produce DNA adducts.

As used herein, the term “chemotherapeutic agent” includes but is not limited to a compound that introduces DNA double strand breaks, for example, bifunctional alkylator, a topoisomerase inhibitor, a monofunctional alkylator, an antimetabolite, a replication inhibitor and a platinum drug. The chemotherapeutic agent as used herein may be temozolomide, etoposide, doxorubicin, gemcitabine, cisplatin or carboplatin.

The term “PARP” as used herein refers to the enzyme family of poly (ADP-ribose) polymerases (PARP). Enzymes of the PARP family include but are not limited to PARP1, PARP2 and PARP3. PARP inhibitors which may be used in accordance with the invention include but are not limited to veliparib, olaparib and talazoparib.

Methods of generating the peptides as described herein are not particularly limiting. Exemplary methods include solid phase peptide synthesis and solution phase peptide synthesis.

Also contemplated are pharmaceutically acceptable salts of the peptide provided herein. The term “pharmaceutically acceptable salt” includes both acid and base addition salts and refers to salts which retain the biological effectiveness and properties of the free bases or acids, and which are not biologically or otherwise undesirable. The pharmaceutically acceptable salts are formed with inorganic or organic acids or bases and can be prepared in situ during the final isolation and purification of the compounds, or by separately reacting a purified compound in its free base or acid form with a suitable organic or inorganic acid or base, and isolating the salt thus formed.

As used herein “pharmaceutical composition” or “composition” refers to a mixture of at least one agent as described herein, one peptide as described herein, or pharmaceutically acceptable salts, solvates, hydrates thereof, with other chemical components, such as pharmaceutically acceptable excipients. Pharmaceutical compositions suitable for the delivery of the agents and peptides as described herein and methods for their preparation will be apparent to those skilled in the art.

Also contemplated are pharmaceutical compositions comprising at least an agent or a peptide provided herein, further comprising one or more chemotherapeutic agent, radiomimetic agent and/or a PARP inhibitor and optionally at least one pharmaceutical excipient. The term “pharmaceutically acceptable excipient” refers to any pharmaceutically acceptable inactive component of the composition. As is known in the art, excipients include diluents, buffers, binders, lubricants, disintegrants, colorants, antioxidants/preservatives, pH-adjusters, etc. The excipients are selected based on the desired physical aspects of the final form: e.g. a parenteral formulation for injection, obtaining a tablet with desired hardness and friability being rapidly dispersible and easily swallowed, and the like. Suitable forms of a pharmaceutical composition may include, but is not limited to, a tablet, capsule, elixir, liquid formulation, delayed or sustained release, and the like. The physical form and/or content of a pharmaceutical composition contemplated are conventional preparations that may be formulated by those skilled in the pharmaceutical formulation field.

A cancer described herein as expressing IGFBP-3, includes a cancer cell population that is tumorigenic, including benign tumours and malignant tumours, or non-tumorigenic, in which at least 5% of the observed cells have the capability of producing the IGFBP-3 protein. Methods of determining IGFBP-3 expression in cancer are not particularly limiting. Exemplary methods include western blotting, immunohistochemistry or immunocytochemistry and PCR (polymerase chain reaction). Exemplary cancers include but are not limited to breast cancer, triple negative breast cancer (TNBC), prostate cancer, pancreatic cancer or glioblastoma cancer.

It is also contemplated that an agent or a peptide provided herein may be delivered to a cancer cell in-vitro or in-vivo. In some embodiments, an agent or a peptide provided herein is administered to an IGFBP-3 expressing cancer cell in-vitro or in-vivo. In certain embodiments, an agent or a peptide provided herein is administered to an IGFBP-3 expressing cancer cell in-vitro or in-vivo and inhibits the NONO-IGFBP-3 interaction in the cancer cell thereof. An agent or peptide provided herein may be administered to a cell with a pharmaceutically acceptable carrier within a composition as herein described.

A “subject” to be treated by a method described herein includes mammal, including a human (“patient”) or non-human subject (for example, cat, dog, and the like). An agent, peptide or composition herein described may be administered to a human or non-human subject. An agent, peptide or composition herein described may be administered to a human cancer cell or a non-human cancer cell in vitro or in vivo. In some embodiments, the cell is a mammalian cell.

As used herein, a “therapeutically effective amount” of an agent, peptide or composition herein described includes an amount, when administered (whether as a single dose or as a time course of multiple treatments), prevents disease advancement or promotes disease regression evidenced by a decrease in severity of disease symptoms, an increase in frequency and duration of disease symptom-free periods, or a prevention of impairment or disability due to the disease affliction. A therapeutically effective amount of an agent, peptide or composition herein described includes a “prophylactically effective amount” which is any amount of an agent, peptide or composition described herein that, when administered to a subject at risk of developing a disease or of suffering a recurrence of disease, inhibits the development or recurrence of the disease. The ability of a therapeutic agent to promote disease regression or inhibit the development or recurrence of the disease may be evaluated using a variety of methods known to the skilled practitioner, such as animal model systems predictive of efficacy in humans, by assaying the activity of the agent in in-vitro assays, or the like. By way of example for the treatment of cancer, a therapeutically effective amount of an agent, peptide or composition as described herein may enhance chemosensitivity or radiosensitivity such that cancer cell growth is reduced by at least about 20%, by at least about 40%, by at least about 60%, or by at least about 80% relative to untreated cancer cells. Alternatively, a therapeutically effective amount of an agent, peptide or composition as herein described may, when used in conjunction with radiotherapy, chemotherapy and/or a PARP inhibitor, allow the dose and/or duration of the radiotherapy, chemotherapy or PARP inhibitor treatment to be decreased while still achieving the same clinical benefit. A therapeutically effective amount of an agent, peptide or composition herein described may enhance chemosensitivity or radiosensitivity such that cancer cell growth is inhibited or reduced to a statistically significant degree of cell growth or tumour growth as compared to control. “Statistical significance” means significance at the p <0.05 level, or such other measure of statistical significance as would be used by those of skill in the art of biomedical statistics in the context of a particular type of treatment or prophylaxis.

Depending upon the cancer type as described herein, the route of administration and/or whether the agent, peptide or composition as herein described is administered locally or systemically, a wide range of permissible dosages are contemplated. The dosages may be single or divided and may be administered according to a wide variety of protocols, including q.d. (once a day), b.i.d. (two times a day), t.i.d. (three times a day), or even every other day, biweekly (b.i.w.), once a week, once a month, once a quarter, and the like. In each of these cases, it is understood that the therapeutically effective amounts described herein correspond to the instance of administration, or alternatively to the total daily, weekly, month, or quarterly dose, as determined by the dosing protocol.

It is contemplated that an agent, peptide or composition as herein described may be administered with one or more chemotherapeutic agent, radiotherapy and/or a PARP inhibitor. Administration as an agent, peptide or composition as herein described with one or more chemotherapeutic agent or PARP inhibitor include but is not limited to simultaneous administration, separate administration or sequential administration. The term “simultaneously” in the context of drug administration refers to an administration of at least 2 active ingredients by the same route and at the same time or at substantially the same time. The term “separately” in the context of drug administration refers to an administration of at least 2 active ingredients at the same time or at substantially the same time by different routes. The term “sequentially” in the context of drug administration refers to an administration of at least 2 active ingredients at different times, the administration route being identical or different. An agent, peptide or composition as herein described can be administered simultaneously with radiotherapy, either before or after radiotherapy.

An agent or composition thereof as described herein may be administered for example orally, intravenously, intramuscularly, intraperitoneally or subcutaneously. A peptide or composition thereof as described herein may be administered intravenously.

### BRIEF DESCRIPTION OF THE FIGURES

Embodiments of the invention will now be described, by way of example only, with reference to the accompanying drawings as follows.

FIG. 1a to 1f: IGFBP-3 forms a complex with NONO in response to etoposide treatment. (a) MDA-MB-468 basal-like TNBC cells were exposed to 20 μM etoposide (Etop) for the indicated times, and NONO was precipitated from cell lysates by anti-IGFBP-3 antiserum (Fab fraction) coupled to agarose beads. Uncoupled agarose beads were used for immunoprecipitation controls. Samples were blotted for NONO after fractionation by SDS-PAGE. Panels on right show blots of whole-cell lysates without immunoprecipitation. Molecular weight markers are shown on the left. (b) IGFBP-3 was downregulated in MDA-MB-468 cells by siRNA, and cell lysates immunoprecipitated with IGFBP-3-Fab beads 2 h after etoposide stimulation. Precipitates were blotted for NONO and IGFBP-3. (c) MDA-MB-468 cells were treated with etoposide, and nuclear extracts were prepared and immunoprecipitated with IGFBP-3-Fab beads. Panels on left show NONO, lamin B1 (nuclear marker), and GAPDH (cytoplasmic marker) in whole nuclear extracts (5% of immunoprecipitated sample). GAPDH in the whole cytoplasmic fraction, run on the same gel, is also shown for comparison. Panels on right show the same proteins after IP. 0 and 4 h time points are shown from a 4-h time-course. For each analyte, all samples (input and IP) were run on the same gel. For NONO, but not lamin B1 or GAPDH, the input blots shown were from shorter exposures, to avoid saturating the images. (d) Similar experiment to that shown in FIG. 1a, but in HCC1806 basal like TNBC cells. (e) Quantitation of bands immunoblotted for NONO in HCC1806 cells. Data are mean band density±SEM from 5 experiments. *P<0.05 vs. time 0 by post hoc Fisher's LSD test after ANOVA. (f) Binding of recombinant IGFBP-3 to immobilized recombinant NONO, measured in an ELISA format in which bound IGFBP-3 is immunodetected and quantitated colorimetrically at 450 nm. Panels show dose—response curves for NONO (left) and IGFBP-3 (right).

FIGS. 2a and 2b: IGFBP-3 complexes with NONO visualized by proximity ligation assay. (a) MDA-MB-468 and (b) HCC1806 TNBC cells were exposed to 20 μM etoposide (Etop) for the indicated times, and bimolecular interactions, shown as red dots, between IGFBP-3 and NONO, as indicated, were measured by PLA. Bar 25 μm.

FIGS. 3a to 3e: PARP inhibition blocks IGFBP-3 complex formation with NONO. (a) MDA-MB-468 cells were incubated for 24 h with 20 μM veliparib (ABT-888) before exposure to 20 μM etoposide. NONO was immunoprecipitated with IGFBP-3-Fab beads and detected by immunoblotting. (b) Quantitation of bands immunoblotted for NONO in MDA-MB-468 cells. Data are mean band density±SEM from 4 experiments. *P<0.05, **P<0.005 vs. the corresponding time 0 by post hoc Fisher's LSD test after ANOVA. NS, not significant. (c) The PARP inhibitor olaparib also inhibits NONO-IGFBP-3 interaction. MDA-MB-468 cells were incubated 24 h with 10 μM olaparib before exposure to 20 μM etoposide. (d) PLA showing interactions (yellow dots) between IGFBP-3 and NONO in MDA-MB-468 cells (above) and HCC1806 cells (below) after 2 h treatment with 20 μM etoposide, following 24 h preincubation±20 μM veliparib. Blue=nuclei (DAPI). Bar 20 μm. Confocal images superimposed over phase contrast images. (e) Quantitation of inhibition by 20 μM veliparib of IGFBP-3 interaction with NONO over 4 h of etoposide treatment in MDA-MB-468 cells, measured by PLA; 5 fields (˜20 nuclei/field) counted for each condition and each timepoint in each experiment. Data are mean values±SEM from 3 experiments. *P<0.001 vs. the corresponding time 0 by post hoc Fisher's LSD test after ANOVA. NS, not significant.

FIGS. 4a to 4f: PARP inhibition decreases DNA end-joining in TNBC cells. (a) Upper panels: γH2AX immunofluorescence in MDA-MB-468 cells at time 0 (i.e. 1 h after exposure to etoposide) and after 4 h of recovery (T4). Cells were pre-treated with olaparib (10 μM) or veliparib (20 μM) as indicated. Bar 20 μm. Lower panels: representative images at higher power of T0 cells±etoposide, to illustrate the punctate γH2AX fluorescence. Bar 10 μm. (b) Mean fluorescence values (arbitrary units)±SEM are shown from 3 experiments. (c) and (d) γH2AX immunofluorescence in HCC1806 cells, with quantitation from 3 experiments, details are as described for panels (a) and (b). Bar 20 μm. ANOVA with Fisher's LSD post hoc LSD test: *(blue) P<0.05 vs. T0+etoposide; *(red) P<0.05 vs. T4 +etoposide; *P<0.05 vs. the corresponding T0 value. (e) DNA end-joining assay: cells were treated with inhibitors (20 μM veliparib or 10 μM olaparib) or no inhibitor (Con) for 24 h, then exposed to 20 μM etoposide for 2 h. In control lanes (right), DNA or nuclear extract (NE) has been omitted. After adding nuclear extract for 30 min, substrate DNA was added and end joining proceeded for 30 min at 25° C. A representative gel is shown for MDA-MB-468 cells. Black arrows show the bands quantitated. Open arrow show size markers in kb. All lanes are from a single gel. (f) Upper panel: Quantitation of end-joining activity 2 h after etoposide in MDA-MB-468 cells, mean±SEM, n=3. Lower panel: Quantitation of end-joining activity 2 h after etoposide in HCC1806 cells, mean±SEM, n=5. *P<0.05 vs. control by post hoc Fisher's LSD test after ANOVA.

FIGS. 5a to 5f: The effects of inhibitory peptides on NONO-IGFBP-3 interaction. (a) NONO-IGFBP-3 binding assay was used to screen inhibitory peptides. The inhibitory effect of peptides #64 to #67 towards the binding of IGFBP-3 with NONO was investigated and compared to no peptide added. The absorbance values are means of duplicate runs in a single assay. (b) Peptide #66 showed a consistent inhibitory effect suggested by significantly reduced absorbance value (P=0.014), indicative of reduced binding between IGFBP-3 and NONO (n=3). (c) and (d) γH2AX signal was measured in 2 BRCA wild-type breast cancer cell lines, MDA-MB-468 and HCC1806, respectively, exposed to 25 μM etoposide, after 16 h incubation with the indicated peptides at 10 μM, or no peptide. Peptides #65 and #66 with overlapping residues HLKFLNVLS (His-Leu-Lys-Phe-Leu-Asn-Val-Leu-Ser) caused a sustained γH2AX signal compared to no peptide or peptide #64 in which a low γH2AX signal was observed. An elevated γH2AX signal is indicative of sustained DNA damage, i.e. inhibition of DNA repair, compared to the control cells. Peptide #66 was further evaluated in the presence of PARP inhibitors, (e) veliparib (ABT-888) and (f) olaparib (Lynparza). The γH2AX signal in the absence of peptide #66 is expressed as 100%, and bars represent the ratio of γH2AX signal in the presence of the peptide, to that in its absence. Preincubation with either PARP inhibitor in the presence of peptide #66 increased the γH2AX signal by almost 2-fold at 10 μM of PARP inhibitor, suggesting a combination effect between the PARP inhibitors and the inhibitor of NONO-IGFBP-3 interaction i.e. peptide #66.

FIGS. 6a and 6b: Peptide #66 inhibits the formation of DNA repair complexes in TNBC cell lines, MDA-MB-468 and HCC1806. (a) In MDA-MB-468 breast cancer cells, exposure to 20 μM of etoposide increased the interaction between IGFBP-3 and NONO/SFPQ, which peaked 2 h after etoposide treatment (black arrow). Treatment with 10 μM of peptide #66 for 1 h prior to etoposide exposure decreased the formation of the IGFBP-3—NONO/SFPQ complex (white arrow). (b) In HCC1806 breast cancer cells, exposure to 20 μM etoposide increased the interaction between IGFBP-3 and NONO/SFPQ, which peaked 2 h after etoposide treatment (black arrow). Treatment with 20 μM of peptide #66 for 1 h prior to etoposide exposure decreased the formation of the IGFBP-3—NONO/SFPQ complex (white arrow).

FIGS. 7a and 7b: Peptide #66 makes breast cancer cells more responsive to the effect of a PARP inhibitor (PARPi) on cell survival following chemotherapy treatment. (a) In HCC1806 breast cancer cells, in the absence of a PARPi, peptide #66 (20 μM) had no effect on cell survival over 14 days (colony number) either before or after etoposide treatment (Etop; 100 nM). In cells treated with a PARPi (veliparib, 5 peptide #66 similarly had no effect on cell survival without etoposide treatment, but after etoposide treatment, peptide #66 significantly decreased cell survival over the effect of the PARPi alone. This indicates that, in the presence of PARPi, peptide #66 can increase cell responsiveness to chemotherapy (etoposide). (a) In the bar graph starting from the left, the 1^{st}, 3^{rd}, 5^{th}, and 7^{th} bars represent conditions without peptide #66 and the 2^{nd}, 4^{th}, 6^{th} and 8^{th} bars represent conditions in the presence of peptide #66. Mean data±SEM from 4 assays, each in triplicate, P<0.001. (b) Representative images of cell colonies after 14 days.

FIGS. 8a and 8b: Peptide #66 diffuses rapidly into the nuclei of breast cancer cells. (a) Fixed cell imaging: peptide #66 (5 μM) labelled biosynthetically with 5-TAMRA (provided by Dr Yu Heng Lau, School of Chemistry, University of Sydney) was added to cultures of HCC1806 breast cancer cells for the indicated times, after which cells were fixed and imaged by confocal microscopy. Images show cell nuclei (blue) and labelled peptide #66 (green); colocalized peptide and nuclei appear cyan, and peaked at 30 min. (b) Live cell imaging: peptide #66 (2.5 μM) labelled biosynthetically with 5-TAIVIRA was added to cultures of HCC1806 breast cancer cells for the indicated times, after which cells were imaged by confocal microscopy without fixation. Images show increasing colocalization of labelled peptide #66 (red) with cell nuclei (blue).

FIGS. 9a and 9b: Peptide #66 inhibits the formation of DNA repair complexes in glioblastoma cell lines, A172 and M059K. (a) In 2 human glioblastoma cell lines, A172 and M059K, treatment with 20 μM of etoposide induced a complex between IGFBP-3 and NONO/SFPQ, determined by coimmunoprecipitation. Complex formation appeared maximal at 2 h in A172 and 1 h in M059K (black arrows). (b) In M059K glioblastoma cells, formation of the complex between IGFBP-3 and NONO/SFPQ peaked at 1 h (black arrow) and was inhibited by incubation with 25 μM of peptide #66 (white arrow).

### DETAILED DESCRIPTION OF PREFERRED EMBODIMENTS

In one embodiment, provided is an agent that inhibits the interaction between IGFBP-3 and NONO. In some embodiments, the agent is a small molecule. In certain embodiments, the agent is a substance or a compound that inhibits the interaction between IGFBP-3 and NONO.

In a further embodiment, provided is an isolated peptide comprising residues:

X₁-X₂-X₃-X₄-X₅-X₆-X₇-X₈-X₉,

wherein X₁ is His, X₂ is Leu, X₃ is Lys, X₄ is Phe, X₅ is Leu, X₆ is Asn, X₇ is Val, X₈ is Leu and X₉ is Ser, or conservative substitutions thereof,

or a pharmaceutically acceptable salt of the peptide.

In some embodiments, provided is a peptide comprising the sequence:

His-Leu-Lys-Phe-Leu-Asn-Val-Leu-Ser-Pro-Arg-Gly,

or conservative substitutions thereof,

or a pharmaceutically acceptable salt of the peptide.

In some embodiments, provided is a peptide comprising the sequence:

Thr-Leu-Asn-His-Leu-Lys-Phe-Leu-Asn-Val-Leu-Ser,

or conservative substitutions thereof,

or a pharmaceutically acceptable salt of the peptide.

In one embodiment, provided is an isolated peptide comprising residues:

X₁-X₂-X₃-X₄-X₅-X₆-X₇-X₈-X₉,

wherein X₁ is His, X₂ is Leu, X₃ is Lys, X₄ is Phe, X₅ is Leu, X₆ is Asn, X₇ is Val, X₈ is Leu and X₉ is Ser, or conservative substitutions thereof,
or a pharmaceutically acceptable salt of the peptide, wherein the peptide inhibits the interaction between IGFBP-3 and NONO.

In some embodiments, provided is an isolated peptide comprising the sequence His-Leu, Leu-Lys, Lys-Phe, Phe-Leu, Leu-Asn, Asn-Val, Val-Leu or Leu-Ser, or conservative substitutions thereof, or a pharmaceutically acceptable salt of the peptide, wherein the peptide inhibits the interaction between IGFBP-3 and NONO.

In some embodiments, provided is an isolated peptide comprising the sequence His-Leu-Lys, Leu-Lys-Phe, Lys-Phe-Leu, Phe-Leu-Asn, Leu-Asn-Val, Asn-Val-Leu, or Val-Leu-Ser, or conservative substitutions thereof, or a pharmaceutically acceptable salt of the peptide, wherein the peptide inhibits the interaction between IGFBP-3 and NONO.

In some embodiments, provided is an isolated peptide comprising the sequence His-Leu-Lys-Phe, Leu-Lys-Phe-Leu, Lys-Phe-Leu-Asn, Phe-Leu-Asn-Val, Leu-Asn-Val-Leu, or Asn-Val-Leu-Ser, or conservative substitutions thereof, or a pharmaceutically acceptable salt of the peptide, wherein the peptide inhibits the interaction between IGFBP-3 and NONO.

In some embodiments, provided is an isolated peptide comprising the sequence His-Leu-Lys-Phe-Leu, Leu-Lys-Phe-Leu-Asn, Lys-Phe-Leu-Asn-Val, Phe-Leu-Asn-Val-Leu, or Leu-Asn-Val-Leu-Ser, or conservative substitutions thereof, or a pharmaceutically acceptable salt of the peptide, wherein the peptide inhibits the interaction between IGFBP-3 and NONO.

In some embodiments, provided is an isolated peptide comprising the sequence His-Leu-Lys-Phe-Leu-Asn, Leu-Lys-Phe-Leu-Asn-Val, Lys-Phe-Leu-Asn-Val-Leu, or Phe-Leu-Asn-Val-Leu-Ser, or conservative substitutions thereof, or a pharmaceutically acceptable salt of the peptide, wherein the peptide inhibits the interaction between IGFBP-3 and NONO.

In some embodiments, provided is an isolated peptide comprising the sequence His-Leu-Lys-Phe-Leu-Asn-Val, Leu-Lys-Phe-Leu-Asn-Val-Leu, or Lys-Phe-Leu-Asn-Val-Leu-Ser, or conservative substitutions thereof, or a pharmaceutically acceptable salt of the peptide, wherein the peptide inhibits the interaction between IGFBP-3 and NONO.

In some embodiments, provided is an isolated peptide comprising the sequence His-Leu-Lys-Phe-Leu-Asn-Val-Leu, or Leu-Lys-Phe-Leu-Asn-Val-Leu-Ser, or conservative substitutions thereof, or a pharmaceutically acceptable salt of the peptide, wherein the peptide inhibits the interaction between IGFBP-3 and NONO.

In some embodiments, provided is an isolated peptide comprising any one or more of the following residues:

X₁, X₂, X₃, X₄, X₅, X₆, X₇, X₈, X₉,

wherein X₁ is His, X₂ is Leu, X₃ is Lys, X₄ is Phe, X₅ is Leu, X₆ is Asn, X₇ is Val, X₈ is Leu and X₉ is Ser, or conservative substitutions thereof,

or a pharmaceutically acceptable salt of the peptide, wherein the peptide inhibits the interaction between IGFBP-3 and NONO.

In some embodiments, provided is a peptide comprising the sequence:

His-Leu-Lys-Phe-Leu-Asn-Val-Leu-Ser-Pro-Arg-Gly,

or conservative substitutions thereof,
or a pharmaceutically acceptable salt of the peptide, wherein the peptide inhibits the interaction between IGFBP-3 and NONO.

In some embodiments, provided is a peptide comprising the sequence:

Thr-Leu-Asn-His-Leu-Lys-Phe-Leu-Asn-Val-Leu-Ser,

or conservative substitutions thereof,
or a pharmaceutically acceptable salt of the peptide, wherein the peptide inhibits the interaction between IGFBP-3 and NONO.

In some embodiments, the peptide of the present disclosure is about 5-50 amino acids in length, such as 5-45 amino acids, 5-40 amino acids, 5-35 amino acids, 5-30 amino acids, 5-25 amino acids, 5-20 amino acids, 5-15 amino acids, or 5-10 amino acids. Preferably, the peptide of the present disclosure is about 9 amino acids, 10 amino acids, 11 amino acids, 12 amino acids, 13 amino acids, 14 amino acids, or 15 amino acids in length. More preferably, the peptide of the present disclosure is about 12 amino acids in length.

In certain embodiments, the peptide of the present disclosure comprises the amino acid sequence Thr-Leu-Asn-His-Leu-Lys-Phe-Leu-Asn-Val-Leu-Ser or a sequence having at least about 80% identity, such as at least about 85% identity, at least about 90% identity or at least about 95% identity to the amino acid sequence Thr-Leu-Asn-His-Leu-Lys-Phe-Leu-Asn-Val-Leu-Ser. In some embodiments, the peptide of the present disclosure comprises the amino acid sequence His-Leu-Lys-Phe-Leu-Asn-Val-Leu-Ser-Pro-Arg-Gly or a sequence having at least about 80% identity, such as at least about 85% identity, at least about 90% identity or at least about 95% identity to the amino acid sequence His-Leu-Lys-Phe-Leu-Asn-Val-Leu-Ser-Pro-Arg-Gly. In some embodiments, the peptide of the present disclosure comprises the amino acid sequence Thr-Leu-Asn-His-Leu-Lys-Phe-Leu-Asn-Val-Leu-Ser-Pro-Arg-Gly or a sequence having at least about 80% identity, such as at least about 85% identity, at least about 90% identity or at least about 95% identity to the amino acid sequence Thr-Leu-Asn-His-Leu-Lys-Phe-Leu-Asn-Val-Leu-Ser-Pro-Arg-Gly. In some embodiments, the peptide of the present disclosure comprises the amino acid sequence His-Leu-Lys-Phe-Leu-Asn-Val-Leu-Ser or a sequence having at least about 80% identity, such as at least about 85% identity, at least about 90% identity or at least about 95% identity to the amino acid sequence His-Leu-Lys-Phe-Leu-Asn-Val-Leu-Ser.

In one embodiment, provided is a pharmaceutical composition comprising an agent of the invention, or an isolated peptide of the invention and optionally at least one pharmaceutically acceptable excipient. In some embodiments, the pharmaceutical composition further comprises a chemotherapeutic agent, a radiomimetic agent or a PARP inhibitor. In a related embodiment, the chemotherapeutic agent is selected from the group consisting of a bifunctional alkylator, a monofunctional alkylator, a topoisomerase inhibitor, an antimetabolite, a replication inhibitor and a platinum drug. In some embodiments, the chemotherapeutic agent is etoposide. In certain embodiments, the PARP inhibitor is veliparib. In some embodiments, the PARP inhibitor is olaparib. In some embodiments, the PARP inhibitor is talazoparib.

In a further embodiment, provided is a method of enhancing chemosensitivity or radiosensitivity in cancer treatment comprising administering to a subject in need thereof a therapeutically effective amount of an agent of the invention, an isolated peptide of the invention or a pharmaceutical composition of the invention, wherein the cancer is an IGFBP-3 expressing cancer. In some embodiments, the IGFBP-3 expressing cancer is breast cancer, prostate cancer, pancreatic cancer or glioblastoma cancer. In certain embodiments, the IGFBP-3 expressing cancer is Triple Negative Breast Cancer (TNBC).

In one embodiment, provided is a method of enhancing chemosensitivity or radiosensitivity in TNBC treatment comprising administering to a subject in need thereof a therapeutically effective amount of an agent of the invention, an isolated peptide of the invention or a pharmaceutical composition of the invention.

In one embodiment, provided is a method of treating cancer comprising administering to a subject in need thereof a chemotherapeutic agent and an agent of the present disclosure or a peptide of the present disclosure. In some embodiments, the cancer of the present disclosure may be mediated by IGFBP-3 and/or NONO/SFPQ. In a related embodiment, the cancer is an IGFBP-3 expressing cancer.

In one embodiment, provided is a method of treating cancer comprising administering to a subject in need thereof radiotherapy and an agent of the present disclosure or a peptide of the present disclosure. In some embodiments, the cancer of the present disclosure may be mediated by IGFBP-3 and/or NONO/SFPQ. In a related embodiment, the cancer is an IGFBP-3 expressing cancer.

In one embodiment, provided is a method of treating cancer comprising administering to a subject in need thereof a radiomimetic agent and an agent of the present disclosure or a peptide of the present disclosure. In some embodiments, the cancer of the present disclosure may be mediated by IGFBP-3 and/or NONO/SFPQ. In a related embodiment, the cancer is an IGFBP-3 expressing cancer.

In one embodiment, provided is a method of inhibiting an interaction between IGFBP-3 and NONO in a cell comprising administering to the cell an agent of the present disclosure or a peptide of the present disclosure. Preferably, the cell is a human cell. More preferably, the cell is in a human body.

In one embodiment, provided is a method of preventing or suppressing DNA DSB repair in a cell comprising administering to the cell an agent of the present disclosure or a peptide of the present disclosure. Preferably, the cell is a human cell. More preferably, the cell is in a human body.

In one embodiment, provided is use of an agent of the invention, or an isolated peptide of the invention in the manufacture of a medicament for enhancing chemosensitivity or radiosensitivity in cancer treatment, wherein the cancer is an IGFBP-3 expressing cancer.

In one embodiment, provided is use of an agent of the invention, or an isolated peptide of the invention in the manufacture of a medicament for enhancing chemosensitivity or radiosensitivity in TNBC treatment.

In some embodiments, provided is an agent of the invention for use in a method of enhancing chemosensitivity or radiosensitivity in cancer treatment, wherein the cancer is an IGFBP-3 expressing cancer. In a related embodiment, the IGFBP-3 expressing cancer is breast cancer, prostate cancer, pancreatic cancer or glioblastoma cancer.

In some embodiments, provided is an agent of the invention for use in a method of enhancing chemosensitivity or radiosensitivity in TNBC treatment.

In certain embodiments, provided is an isolated peptide of the invention or a pharmaceutically acceptable salt thereof for use in a method of enhancing chemosensitivity or radiosensitivity in cancer treatment, wherein the cancer is an IGFBP-3 expressing cancer. In a related embodiment, the IGFBP-3 expressing cancer is breast cancer, prostate cancer, pancreatic cancer or glioblastoma cancer.

In some embodiments, provided is an isolated peptide of the invention or a pharmaceutically acceptable salt thereof for use in a method of enhancing chemosensitivity or radiosensitivity in TNBC treatment.

Further preferred embodiments of the invention will now be described, by way of example only, with reference to the accompanying drawings.

### EXAMPLES

General Material

Etoposide was obtained from Sigma-Aldrich (St. Louis, Mo., USA). Veliparib (ABT-888) was from Selleckchem, Houston, Tex., USA and olaparib from AdooQ Bioscience, Irvine, Calif. Rabbit antiserum R-100 against full-length human IGFBP-3, and recombinant human IGFBP-3 expressed in human cells, were prepared in-house. Recombinant human NONO, Myc-DDK tagged (TP326567) was obtained from Origene, Rockville, Md., USA. FLAG antibody plates (L00455C) were from GenScript, Piscataway, N.J., USA. Goat anti-rabbit IgG-HRP (ab97080) was from Abcam, Melbourne, VIC, Australia, and 1-Step Turbo TMB-ELISA substrate solution was from ThermoFisher, Scoresby, VIC, Australia.

Cell Culture

The human basal-like triple negative breast cancer (TNBC) cell lines MDA-MB-468 and HCC1806 were obtained from ATCC, Manassas, Va. and maintained in RPMI 1640 medium containing 5% FBS and 10 μg/mL bovine insulin under standard conditions. Cryopreserved stocks were established within 1 month of receipt, and fresh cultures for use in experiments were established from these stocks every 2 to 3 months. All cell lines tested negative for mycoplasma. Inhibitor treatments were carried out for 24 h with veliparib (20 μM), olaparib (10 μM), followed by etoposide (20 μM).

siRNA Mediated Transient Knockdown

IGFBP-3 was downregulated using siRNAs from Qiagen (Hilden, Germany) (Table 1). Transfection was performed by electroporation (Amaxa Nucleofector, Lonza, Cologne, Germany). In brief, the cells were harvested by trypsinization and resuspended at 1×10⁶ cells in 100 μL Transfection Reagent solution V (Lonza) and mixed with 100 Nm targeting siRNA or AllStars negative control siRNA (Qiagen). Immediately after electroporation, cells were transferred to complete medium and plated for analysis. Knockdown was confirmed by qRT-PCR as previously described (Martin J L et al., Mol Cancer Therap., 2014, 13, 316-328) using Taqman probe Hs00181211_m1 for IGFBP-3 and hydroxymethylbilane synthase (HMBS; Hs00609297_m1) as an internal control (Applied Biosystems, Foster City, Calif., USA).

TABLE 1
IGFBP-3 siRNAs

| IGFBP-3 | Sense strand |
|---|---|
| siRNA #1^{a} | 5′GUU GAC UAC GAG UCU CAG AUU 3′ |
| siRNA #2^{b} | 5′AGG UUA AUG UGG AGC UCA AUU 3′ |

^{a}Designed by Qiagen (Hilden, Germany)
^{b}Catalog No. SI02780589, Qiagen

Co-Immunoprecipitation and Western Blotting

Immunoprecipitation of IGFBP-3 complexes using anti-IGFBP-3 IgG (Fab fraction) coupled to agarose beads was performed as previously described (Lin M Z et al., Oncogene, 2014, 33, 85-96). For immunoprecipitations using NONO, cells (˜1×10⁶) were lysed in 1 mL ice-cold RIPA lysis buffer (50 mM Tris-HCl pH 7.4, 150 mM NaCl, mM EDTA, 1% Triton X-100) supplemented with protease (cOmplete™ Mini) and phosphatase (PhosSTOP™) inhibitors (Roche; Sigma-Aldrich, Sydney, Australia) at 4° C. for 1 h and spun at 10,000×g for 10 min to pellet cell debris. Lysates were precleared by mixing with 20 μL of Protein A agarose beads (Roche; Sigma-Aldrich) for 1 h at 4° C. Pre-cleared lysates were mixed overnight with specific antibodies and Protein A agarose beads (blocked by mixing with 1% BSA in RIPA buffer for 1 h at 4° C.). The antibody used for IP was NONO [N-terminal] (Sigma-Aldrich #N8789), 2.5 μg per sample. To prepare nuclear extracts for coIP, cellular fractionation was performed according to the manufacturer's protocol for the NE-PER Nuclear and Cytoplasmic Extraction Kit (ThermoFisher). Immunoprecipitated samples were resuspended in Laemmli sample buffer containing 50 mM dithiothreitol, heated at 95-100° C. for 6 min, and fractionated on 12% SDS-PAGE gels. Proteins were transferred to Protran® supported nitrocellulose membranes (Amersham, UK) at 160 mA for 2 h. Membranes were blocked in 50 g/L skim milk powder and probed with primary antibodies (NONO (as above), 1:2000; IGFBP-3 [C19], 1:750, Santa Cruz Biotechnology #sc-6003; GAPDH [14C10], 1:2000, Cell Signaling #2118; and Lamin B1, 1:2000, Abcam #ab16048) at 4° C. for 16 h. Immunoreactive bands were visualized as previously described (Lin M Z et al., Oncogene, 2014, 33, 85-96).

Proximity Ligation Assay (PLA)

PLA was performed using the Duolink Detection Kit (Olink Bioscience Uppsala, Sweden) as previously described (Lin M Z et al., Oncogene, 2014, 33, 85-96). Briefly, cells were grown on 8-mm glass coverslips to 50% confluency, treated, and prepared for microscopy by fixing, permeabilizing and blocking. Coverslips were incubated with primary antibody pairs (raised in different species) targeting the proteins under investigation overnight at 4° C. 1:500; NONO (as above) and 1:500; IGFBP-3 (as above), 1:100. This was followed by incubation with PLA probes MINUS and PLUS for 1 h at 37° C., probe ligation for 30 min at 37° C. and amplification over 100 min at 37° C. Interactions were detected as amplified far-red signals using a Leica TCS SP5 confocal microscope (Leica Microsystems, Wetzlar, Germany) and quantitated using Image J software.

γH2AX Immunofluorescence

Cells grown on 8-mm glass coverslips were washed three times with PBS, fixed with 4% paraformaldehyde for 15 min, permeabilized with 0.2% Triton X-100 for 5 min and blocked with 2% BSA for 1 h. Cells were then incubated with rabbit anti-phospho-histone γH2A.X (Ser139) (1:200; Cell Signaling Technology, #9718) overnight at 4° C., washed, and further incubated with anti-rabbit secondary antibody, tagged with Alexa Fluor 594 (Life Technologies, Carlsbad, Calif., USA). For controls, cells were treated with isotype-matched IgG from the same species. Slides were mounted using ProLong Gold Antifade Reagent (Life Technologies). Fluorescence images were captured by confocal laser scanning microscope. γH2AX fluorescence was quantitated in 5-6 fields for each condition using ImageJ (NIH, Bethesda, Md.), and corrected for the number of nuclei per field (average=14), visualized by DAPI staining. Data were calculated from three replicate experiments.

Discovery of IGFBP-3-Interacting Proteins

MDA-MB-468 cells were grown to 90% confluence in T75 flasks in RPMI 1640 medium containing 5% fetal calf serum and 10 μg/mL bovine insulin, then exposed to 20 μM etoposide, or medium alone for control cells, for 2 h. Medium was removed, and cells were washed twice in PBS, then lysed with 1 mL ice-cold RIPA buffer supplemented with protease and phosphatase inhibitors (as above) at 4° C. for 30 min. After centrifugation to remove insoluble material, the supernatant was incubated overnight with anti-IGFBP-3 IgG (Fab fraction) conjugated to agarose beads as previously described (Lin M Z et al., Oncogene, 2014, 33, 85-96). Control precipitations used agarose beads without antibody. Beads were pelleted by centrifugation, washed 4 times in ice-cold PBS, resuspended in 50 μL 0.1% solution of RapiGest SF surfactant (Waters, Rydalmere, NSW, Australia) in 20 mM Tris-HCl buffer, pH 7.4. After boiling for 5 min to dissociate immunoprecipitated proteins, supernatants were collected by centrifugation and stored at −80° C. before analysis. For proteomic analysis, tris(2-carboxyethyl) phosphine was added to 5 mM final concentration, samples were heated at 60° C. for 30 min, then cooled to room temperature. Iodoacetamide was added to 15 mM and reacted for 30 min in the dark. Trypsin Gold (MS grade; Promega, Alexandria, NSW, Australia) was added at 1:50 by protein weight, the solutions were incubated overnight at 37° C., and TFA was added to 0.5% final. After 45 min at 37° C., samples were immersed in liquid nitrogen to precipitate the RapiGest, then centrifuged for 10 min, and the supernatants collected. Samples were fractionated on an UltiMate 3000 nanoLC (Thermo Scientific) and spotted onto a Bruker MTP 384 AnchorChip target plate (Bruker, Preston, VIC, Australia) using a Proteineer fc II fraction collector (Bruker) as described previously (Hunt N J., J Proteom., 2016, 138, 48-60). MS/MS data were acquired on an UltrafleXtreme MALDI TOF/TOF mass spectrometer (Bruker) with a smart beam laser run at 2 kHz, with data processing and peptide identification performed as previously described (Hunt N J., J Proteom., 2016, 138, 48-60).

NONO-IGFBP-3 Binding Assay

NONO was diluted in 50 mM sodium phosphate, 0.05% BSA, pH 7.4, and incubated 16 h at indicated concentrations in wells of FLAG (i.e. DDK) antibody plates. All incubations were at 22° C. in 100 μL of 0.1 M Tris-HCl, 0.05% BSA, pH 7.4 (incubation buffer) unless noted otherwise. After 4 washes with 250 μL cold incubation buffer, wells were incubated for 2 h at 22° C. with recombinant human IGFBP-3 at indicated concentrations in incubation buffer containing 1% BSA. After 4 washes as above, wells were incubated 2 h with anti-human IGFBP-3 antiserum R-100 at 1:25,000, washed 4 times, incubated 1 h with goat anti-rabbit IgG-HRP at 1:20,000, washed 4 times, and incubated 30 min with 100 μL TMB solution. Reactions were stopped by adding 100 μL 1 M H₂SO₄ and absorbance read at 450 nm.

DNA End-Joining Assay

Nuclear extraction and end-joining assay was performed as previously described (Andrin C et al., J Blot Chem., 2004, 279, 25017-25023; Andrin C et al., Nucleus., 2012, 3, 384-395) with slight modifications. Briefly, HCC1806 cells were grown in flasks and treated with inhibitors for 24 h followed by etoposide treatment for 2 h as described above. After isolation of nuclei by centrifugation through a buffer containing 300 mM sucrose, the washed nuclear pellet was extracted into high-salt buffer (20 mM Hepes, pH 7.5, 25% glycerol, 420 mM NaCl, 0.2 mM EDTA, 1.5 mM MgCl2) for 30 min on ice, and insoluble material was removed by centrifugation. The soluble nuclear extract was used in the end-joining assay. Restriction enzymes NheI and EcoRI (New England Biolabs, Ipswich, Mass., USA) were used to digest a EGFP-C1 plasmid (Clontech, Mountain View, Calif., USA) to generate a DNA fragment of 4 kb with non-homologous ends. The linearized plasmid was separated by 0.8% agarose gel electrophoresis, purified using a DNA gel extraction kit (Qiagen), and used as the substrate for end-joining assays. Nuclear extract (2 μg) was mixed with end-joining assay buffer (7.5 mM Tris pH 8.0, 0.2 mM CaCl₂, 10 mM MgCl₂, 50 mM KCl, 1.2 mM ATP and 0.5 mM DTT) and allowed to stand for 30 min at 22° C. Repair was initiated by adding 100 ng of prepared linearized DNA and incubated at 25° C. for 30 min, stopped by the addition of 0.5 M EDTA, 0.5% SDS and 10 mg/mL Proteinase K. DNA bands were separated on a 0.7% agarose gel, stained with SYBR Gold (Life Technologies), and visualized on a BioRad ChemiDoc imaging system.

Generation and Testing of Inhibitory Peptides

A library of 85 overlapping 12-residue peptides covering the full-length sequence of mature human IGFBP-3 (264 residues) was synthesised and purified to at least 80% purity by ChinaPeptides Co., Shanghai, China. The overlap was nine residues, i.e. residues 1-12, 4-15, . . . 250-261, 253-264. For each peptide, 5 mg (calculated as approx. 3.79 μmol) was dissolved in 379 μl of 20% acetonitrile in water, to give a concentration of 10 mM. For screening assays, NONO was bound to each well at 240 ng/100 μl. After 16 h incubation and washing as described above, the IGFBP-3 peptides, diluted 1:500 to 20 μM in incubation buffer, were added diluted 1:1 with recombinant IGFBP-3 (25 ng) in a total volume of 100 μl incubation buffer. The final peptide concentration was 10 μM and the final IGFBP-3 concentration approx. 6 nM. After 2 h incubation the IGFBP-3 binding was determined as described above.

Epitope Mapping

To further refine the amino acid residues of IGFBP-3 involved in the interaction between IGFBP-3 and NONO, three additional derivatives of the peptide His-Leu-Lys-Phe-Leu-Asn-Val-Leu-Ser-Pro-Arg-Gly were synthesized and tested:
(1A): His-Ala-Lys-Phe-Ala-Asn-Val-Ala-Ser-Pro-Arg-Gly, in which the three Leu residues were changed to Ala
(2A): His-Leu-Ala-Phe-Leu-Asn-Val-Leu-Ser-Pro-Ala-Gly, in which the two basic amino acids Lys and Arg were both changed to Ala
(3A): His-Leu-Lys-Phe-Leu-Asn-Val-Leu-Ser, in which the three carboxyterminal residues were deleted.

Each peptide was tested for its inhibitory activity in the cell-free IGFBP-3-NONO binding assay and the cell-based co-immunoprecipitation assay, and, if found to be inhibitory, was further evaluated for its effect on γH2AX immunofluorescence following etoposide treatment of cells.

Effects of Peptide on the γH2AX Response to Etoposide in Breast Cancer Cells
MDA-MB-468 or HCC1806 human TNBC cells were preincubated for 24 h with PARP inhibitors olaparib or veliparib at 0, 1 or 10 μM, without or with 10 μM peptide. Etoposide (at the indicated final concentration) was added for 1 h, then cell lysates were harvested in Laemmli buffer, separated by SDS-PAGE, and blotted for γH2AX.

Statistics

ANOVA with post hoc Fisher's LSD test (SPSS v.22 for Mac; IBM Corp, Armonk, N.Y., USA) was used for multiple group comparisons.

### Example 1

### NONO Interacts with IGFBP-3

An unbiased proteomic screen for proteins that interact with IGFBP-3 2 h after etoposide treatment was carried out. Examination by LC-MALDI-TOF/TOF mass spectrometry of proteins co-precipitating with IGFBP-3 from whole cell lysates consistently revealed NONO as a putative IGFBP-3 binding partner. Unique peptides for the NONO protein, identified by mass spectrometry from IGFBP-3-coimmunoprecipitation (coIP) experiment are shown in Table 2.

TABLE 2
Unique NONO peptides identified by LC-MALDI-TOF/TOF from IGFBP-3
co-immunoprecipitation experiments

| | | | | Δ m/z | | |
|---|---|---|---|---|---|---|
| Accession | Range | m/z meas. | Mr calc. | [ppm] | Sequence | Modifications |
| NONO_HUMAN | 143-153 | 1248.6073 | 1427.6081 | −6.49 | R.FACHSASLTVR.N | Carbamidomethyl: 3 |
| NONO_HUMAN | 191-202 | 1231.6920 | 1230.6721 | 10.25 | K.GIVEFSGKPAAR.K | |
| NONO_HUMAN | 357-365 | 1263.5989 | 1262.5972 | −4.45 | R.RQQEEMMRR.Q | |
| NONO_HUMAN | 365-378 | 1636.8313 | 1635.8118 | 7.48 | R.RQQEGFKGTFPDAR.E | |
| NONO_HUMAN | 366-378 | 1480.7236 | 1479.7106 | 3.84 | R.QQEGFKGTFPDAR.E | |
| NONO_HUMAN | 384-398 | 1538.6691 | 1537.6622 | −0.23 | R.MGQMAMGGAMGINNR.G | |
| NONO_HUMAN | 435-456 | 2163.0680 | 2162.0579 | 1.31 | R.FGQAATMEGIGAIGGTPPAFNR.A | |
| NONO_HUMAN | 435-468 | 3372.8220 | 3371.6833 | 38.96 | R.FGQAATMEGIGAIGGTPPAFNRAA | |
| | | | | | PGAEFAPNKR.R | |
| NONO_HUMAN | 457-468 | 1228.8834 | 1227.6360 | 18.35 | R.AAPGAEFAPNKR.R | |
| NONO_HUMAN | 457-469 | 1384.7644 | 1383.7371 | 14.40 | R.AAPGAEFAPNKRR.R | |

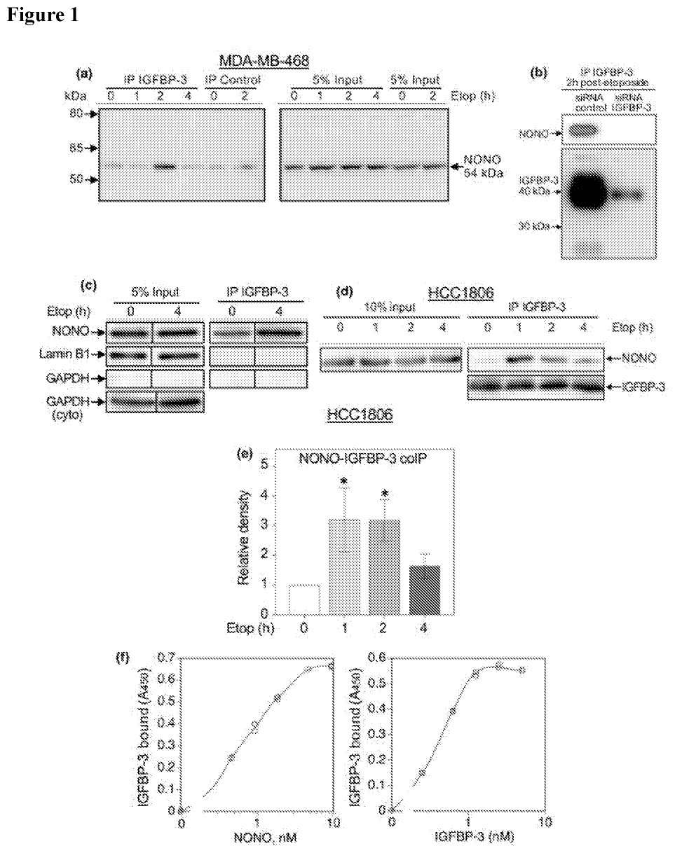

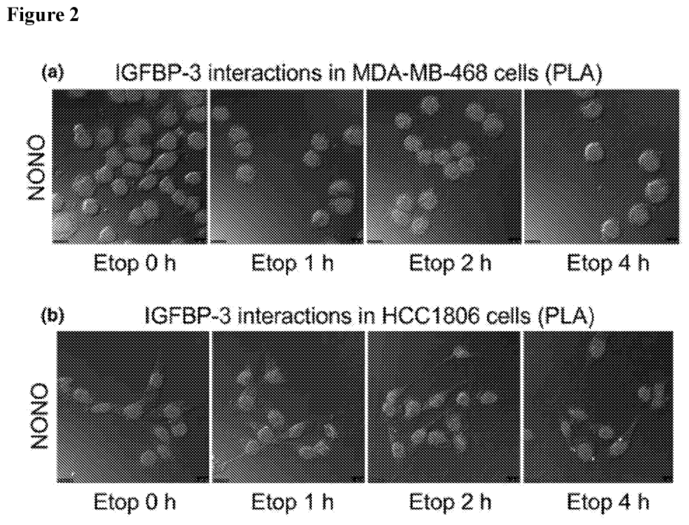

The interaction, and its stimulation by chemotherapy treatment, were confirmed by coIP and western blotting (FIG. 1), and by proximity ligation assay (PLA, FIG. 2). FIG. 1a shows western blots of whole cell lysates from MDA-MB-468 cells treated with etoposide for 0 to 4 h, after immunoprecipitation using agarose-immobilized anti-human IGFBP-3 IgG (Fab fragment) or control, non-immune agarose beads. CoIP of NONO typically peaked after 2 h exposure to etoposide, although the time-course was variable among experiments, with earlier (1 h) or later (4 h) peaks seen in some experiments. This variability may be related to the passage number of the cells, with the peak time tending to increase with extended passages after thawing. Weak bands for both antigens were also seen in some control IPs.

When IGFBP-3 was downregulated transiently in MDA-MB-468 cells by siRNA, the amount of NONO detectable after IP with anti-human IGFBP-3, 2 h after etoposide treatment, was greatly reduced compared to that from cells treated with control non-silencing siRNA (FIG. 1b) thus providing further support that NONO was precipitating in a complex containing IGFBP-3. Immunoprecipitated IGFBP-3 was detected as a diffuse band around 40 kDa (known to be a mixture of glycosylation isoforms) plus a weak band, probably proteolyzed IGFBP-3, below 30 kDa. An increase in IGFBP-3-associated NONO after etoposide treatment was similarly observed in isolated nuclear extracts rather than whole cell lysates (FIG. 1c). Similar to MDA-MB-468 cells, IGFBP-3-associated NONO also increased in HCC1806 basal-like breast cancer cells in response to etoposide, typically peaking 1-2 h after etoposide treatment (FIG. 1d). A representative image of immunoprecipitated IGFBP-3, measured in most coIP experiments, is also shown in FIG. 1d. In FIG. 1e, the association of NONO with IGFBP-3 in HCC1806 cells is quantitated for 5 experiments, the broad peaks representing the somewhat variable time-courses. IGFBP-3 interaction with NONO was also demonstrated in a cell-free system. This was examined using a direct binding assay in which IGFBP-3 bound to immobilized NONO was detected in an ELISA format. FIG. 1f shows dose-response curves for a fixed IGFBP-3 concentration (10 ng/100 μL; approx. 2.5 nM) bound to increasing concentrations of immobilized NONO, and for increasing IGFBP-3 concentrations bound to a fixed amount of NONO (25 ng/100 μL; approx. 4.6 nM). The NONO-IGFBP-3 interaction appears dose dependent and saturable, consistent with NONO forming a specific protein—protein interaction with IGFBP-3.

FIG. 2 confirms the association of NONO with IGFBP-3 in breast cancer cells by proximity ligation assay (PLA). Biomolecular interaction between IGFBP-3 and NONO was minimal before etoposide treatment, typically peaking 2 h after exposure to 20 μM etoposide and decreasing again at 4 h. In control PLA experiments, in which either detection antibody was omitted, no signal was observed (not shown). These independent approaches confirm that NONO forms transient nuclear complexes with IGFBP-3 in basal-like TNBC cells treated with etoposide.

### Example 2

### The Effects of PARP Inhibition on IGFBP-3 Interaction with NONO

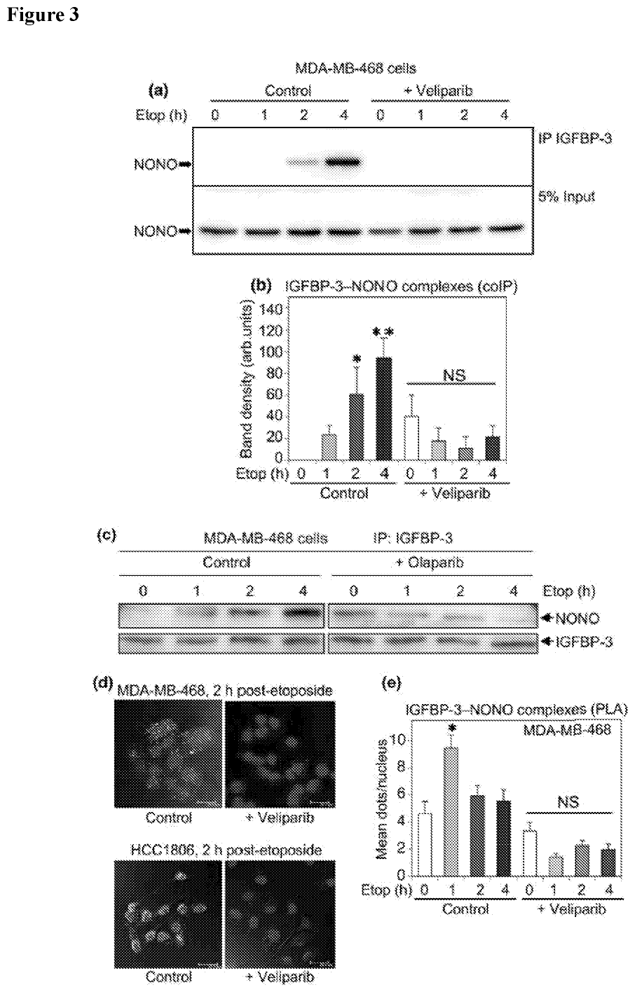

Since NONO recruitment to DNA damage sites is reported to be PARP-dependent (Krietsch J., Nucl Acids Res., 2012, 40, 10287-10301), we examined the effect of PARP inhibition on the interaction between IGFBP-3 and NONO. FIG. 3a shows in MDA-MB-468 cells that IGFBP-3 complexes with NONO, determined by immunoblotting after coIP, were abolished if cells were preincubated with the PARP1 and PARP2 inhibitor veliparib (20 μM) for 24 h prior to exposure to etoposide. Data for 3 experiments in MDA-MB-468 cells shown in FIG. 3b for IGFBP-3-NONO interactions. A similar inhibitory effect was seen after preincubation with a second PARP inhibitor, olaparib at 10 μM (FIG. 3c). The inhibitory effect of veliparib on complex formation was confirmed by PLA in both MDA-MB-468 and HCC1806 cells (FIG. 3d), showing the increase in IGFBP-3-NONO complexes 2 h after etoposide treatment was abolished by preincubation with 20 μM veliparib. FIG. 3e shows the quantitation of 3 replicate experiments in MDA-MB-468 cells, with the effect of veliparib highly significant by ANOVA (P<0.001). Therefore, the formation of EGFR-dependent complexes between IGFBP-3 and NONO in basal-like TNBC cell lines exposed to DNA-damaging chemotherapy requires PARP activity.

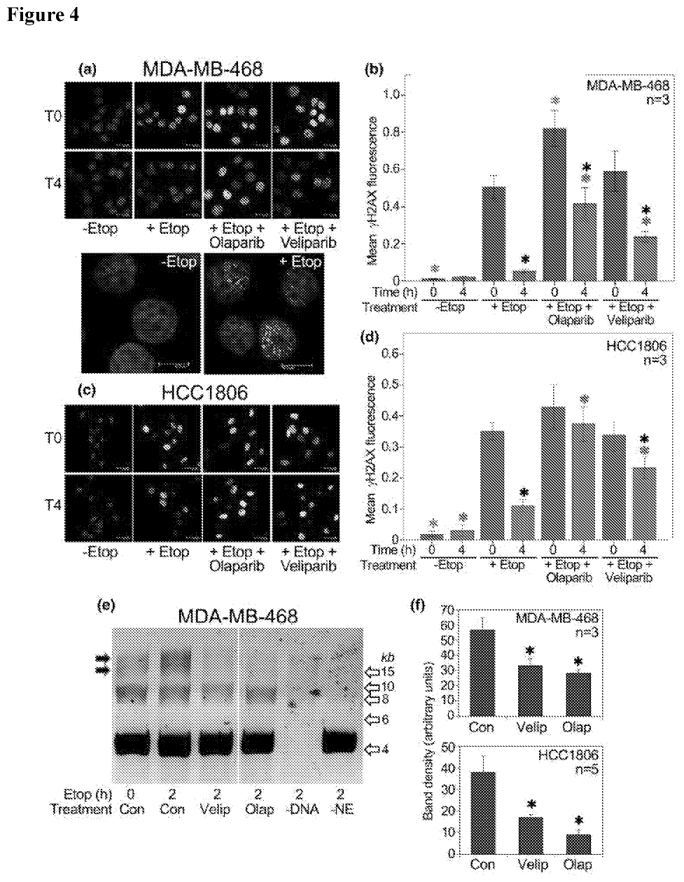

Consistent with the above, DNA repair activity in TNBC cell lines was inhibited by PARP inhibitors. As shown in FIG. 4a, c, treatment of either MDA-MB-468 or HCC1806 cells with etoposide for 1 h (T0) caused a significant increase in foci of histone H2AX phosphorylated on serine 139 (γH2AX), which accumulates at sites of DNA double-strand breaks. This signal had substantially declined after 4 h (T4), consistent with DNA repair over this period. The addition of either PARP inhibitor, olaparib or veliparib, significantly prevented the loss of γH2AX signal, indicating that both drugs were inhibitory to DSB repair in these cell lines, neither of which has a mutation in BRCA1 or BRCA2. Data for both cell lines are quantitated in FIG. 4b, d. Etoposide treatment also increased activity in a direct DNA end-joining assay using nuclear extracts from treated cells (FIG. 4e). In extracts from cells treated with either PARP inhibitor, end joining activity was inhibited by approximately 50% in both MDA-MB-468 and HCC1806 cells, as shown quantitatively in FIG. 4f.

### Example 3

### Inhibition of NONO-IGFBP-3 Interaction

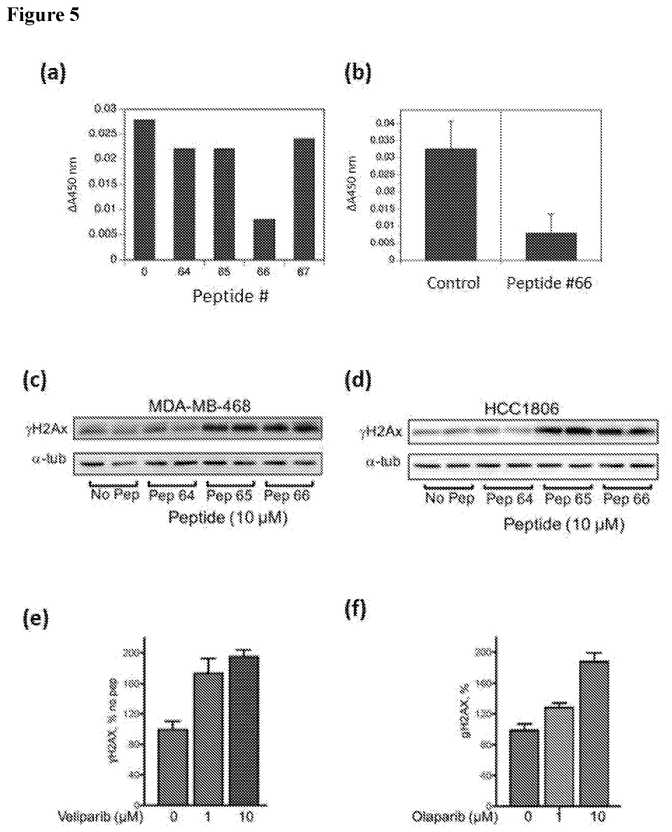

Peptide #66 consistently inhibited IGFBP-3 binding to NONO in the screening assay. This peptide has the sequence HLKFLNVLSPRG (i.e. His-Leu-Lys-Phe-Leu-Asn-Val-Leu-Ser-Pro-Arg-Gly). FIG. 5a shows results from a NONO-IGFBP-3 binding assay comparing peptides #64 to 67 to no added peptide. FIG. 5b shows the mean inhibitory effect of peptide #66 on the NONO-IGFBP-3 interaction from 3 assays, indicated by the lower absorbance value.

### Example 4

### Sustained DNA Damage

FIG. 5c and FIG. 5d shows γH2AX signals in two BRCA wild-type breast cancer cell lines MDA-MB-468 and HCC1806, respectively, exposed to 25 μM etoposide, after 16 h incubation with the indicated peptides #64 to #66 at 10 μM, or no peptide. In this experiment, peptides #65 (TLNHLKFLNVLS) and #66 (HLKFLNVLSPRG) with overlapping residues HLKFLNVLS (i.e. His-Leu-Lys-Phe-Leu-Asn-Val-Leu-Ser) caused a sustained γH2AX signal compared to no peptide or peptide #64 (MEDTLNHLKFLN) in which a low γH2AX signal was seen. An elevated γH2AX signal is indicative of sustained DNA damage (i.e. inhibition of DNA repair) compared to the control cells. Peptide #66 was also evaluated in the presence of PARP inhibitors, veliparib (ABT-888) and olaparib (Lynparza). In FIG. 5e and FIG. 5f, the γH2AX signal in the absence of peptide #66 is expressed as 100%, and bars represent the ratio of γH2AX signal in the presence of the peptide, to that in its absence. Preincubation with either PARP inhibitor in the presence of peptide #66 increased the γH2AX signal by almost 2-fold at 10 μM of PARP inhibitors (FIG. 5e: veliparib; FIG. 5f: olaparib), suggesting a combination effect between the PARP inhibitor and the inhibitor of NONO-IGFBP-3 interaction i.e. peptide #66. These results demonstrate that inhibition of NONO-IGFBP-3 interaction can act in conjunction with PARP inhibition as an effective means to inhibit DNA damage repair following exposure to a DNA-damaging chemotherapy drug such as etoposide.

### Example 5

### Peptide #66 Inhibits the Complex Formation Between IGFBP-3 and NONO/SFPQ in TNBC Cell Lines

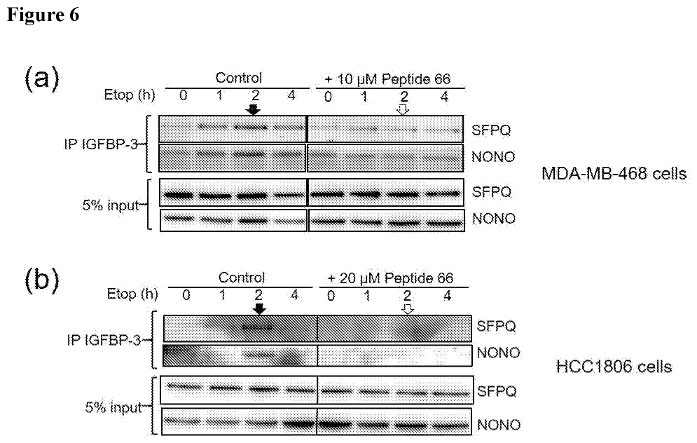

Using TNBC cells lines as described above, MDA-MB-468 and HCC1806, it was demonstrated that peptide #66 inhibited the complex formation between IGFBP-3 and NONO/SFPQ (FIG. 6). MDA-MB-468 basal-like TNBC cells were incubated for 1 h without (control) or with 10 μM of peptide #66 prior to exposure to 20 μM etoposide (Etop) for the indicated times (0, 1, 2, or 4 h), after which NONO and SFPQ were precipitated from cell lysates by anti-IGFBP-3 antiserum (Fab fraction) coupled to agarose beads (FIG. 6a). The samples were blotted for NONO or SFPQ after fractionation by SDS-PAGE. HCC1806 basal-like TNBC cells were treated and analysed as described in above, except that the concentration of peptide #66 was 20 μM (FIG. 6b). Preincubation for 1 h with peptide #66 inhibited the subsequent formation of complexes between IGFBP-3 and NONO or SFPQ after etoposide exposure, as indicated by the decreased density of bands detected by immunoblotting (FIG. 6). The inhibition of complex formation after etoposide exposure indicates that the repair of DNA damage, caused by the chemotherapy agent (Etop), is impaired in the presence of peptide #66.

### Example 6

### Peptide #66 Makes Breast Cancer Cells More Responsive to the Effect of a PARPi

In a 14-day colony formation or clonogenic survival assay, it was shown that the survival of HCC1806 breast cancer cells for 12 days after a 2-day exposure to a low concentration of etoposide (100 nM), was inhibited maximally by a combination of a PARPi (veliparib) and peptide #66. HCC1806 cells (500 cells/well) were plated in 6-well plates for 24 h prior to being treated with or without 5 μM of PARPi (veliparib) for a further 24 h. Cells were then exposed to 20 μM of peptide #66 (or not, as indicated) for 1 h, followed by 100 nM of etoposide treatment (or not, as indicated) for 48 h, after which the medium was replaced with fresh medium. Colony formation was observed for a further 12 days during which cells were refreshed with new media every 3 days. Colonies were washed with PBS and stained using 0.5% Crystal Violet (Sigma Aldrich) in 20% methanol for 30 min prior to rinsing with water. Colonies, defined as clusters of at least 30 cells, were imaged and counted with an AID vSpot Spectrum imager (AutoImmun Diagnostika GmbH, Strassberg, Germany).

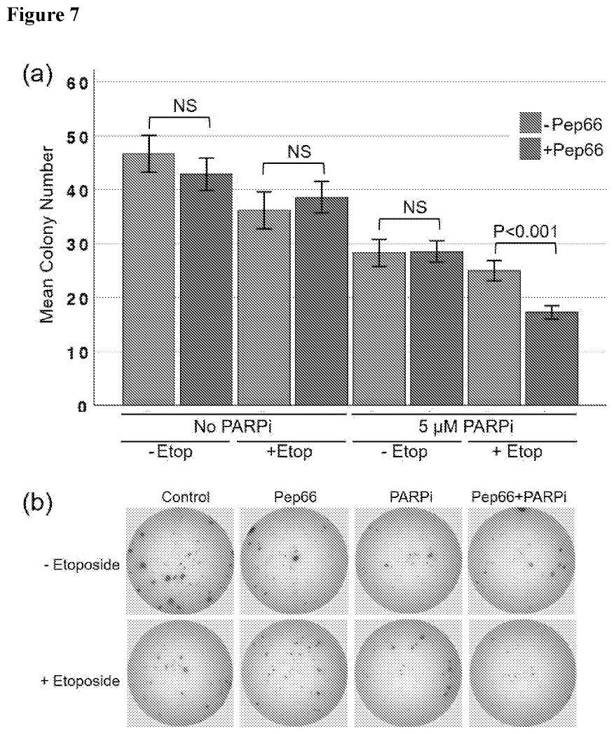

The 14-day colony formation or clonogenic survival assay measures the ability of cells to survive 2 days of chemotherapy-induced DNA damage, and form colonies of at least 30 cells over the next 12 days. The etoposide concentration used was very low (100 nM), so that only minor cell death would occur under control conditions. The purpose was to find conditions under which the cells become more sensitive to this low dose of chemotherapy. In the absence of etoposide, the PARP inhibitor veliparib, at the concentration used (5 inhibited cell survival by about one-third, and peptide #66 had no additional effect (FIG. 7). In contrast, in cells exposed to etoposide, the PARP inhibitor again inhibited cell survival by about one-third, but the addition of peptide #66 caused highly significant further inhibition of cell survival (colony formation). This experiment provides a proof of principle that peptide #66 can act in combination with PARP inhibition to enhance the ability of chemotherapy to inhibit breast cancer cell survival.

### Example 7

### Peptide #66 Rapidly Enters the Nucleus of Breast Cancer Cells

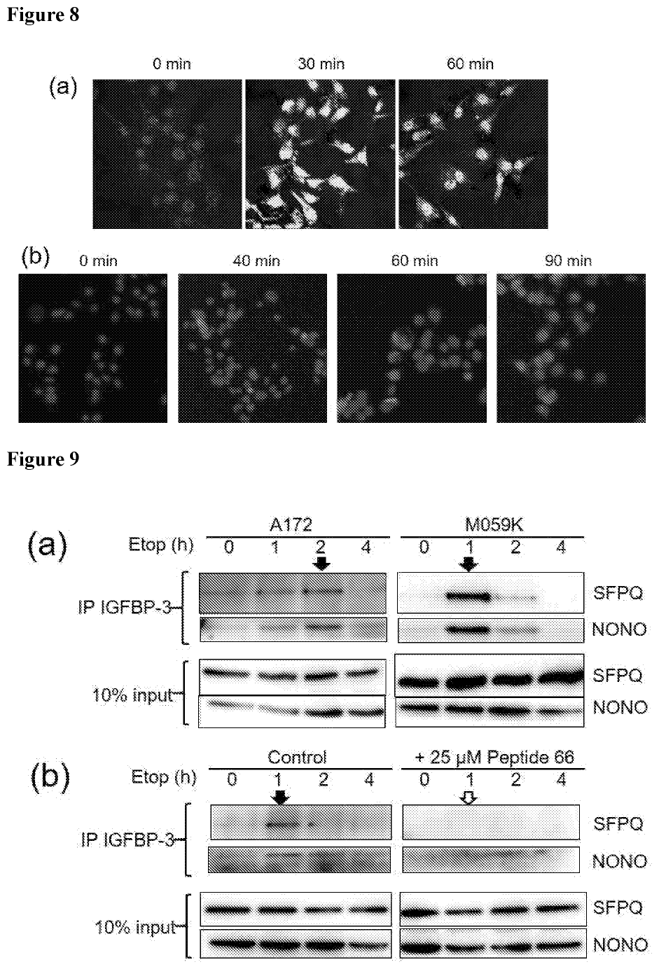

Fluorescently-labelled peptide #66 was used to demonstrate that the peptide can directly diffuse into the cell nuclei. The peptide was synthesised with the fluorescent dye 5-TAMRA (5-carboxytetramethyl-rhodamine) covalently bound at its amino-terminus. Since under some circumstances the fixation of cells prior to imaging may introduce artefacts, experiments were performed to detect the localisation of the peptide both with and without fixation of the cells. Fixed cell imaging (FIG. 8a): HCC1806 cells were grown on 8 mm glass coverslips; 10 μM of peptide #66 labelled with 5-TAMRA was mixed with complete growth medium containing serum, and directly added to the cells. At the indicated time points, cells were washed three times with PBS, fixed with 4% paraformaldehyde for 15 min, washed three times and mounted on slides using ProLong Gold Antifade Reagent containing the nuclear stain DAPI (Life Technologies). Confocal fluorescence images were captured with a Leica TCS SP5 confocal laser scanning microscope (Leica Microsystems, Wetzlar, Germany). Live cell imaging (FIG. 8b): HCC1806 cells were plated in an 8-well chamber slide (Nunc Lab-Tek, Sigma Aldrich); 0.1 mg/mL Hoechst nuclear stain (Life Technologies) was added to the cells for 15 mins and imaged (0 min time point) using Leica TCS SP5 confocal microscope. The medium was then changed to complete growth medium containing serum plus 10 μM of peptide #66 labelled with 5-TAMRA. Images were taken at the times indicated.

Fixed cell imaging showed rather diffuse green staining associated with the cells after 30 min, strongly associated with cell nuclei as indicated by the cyan colour of the merged nuclei (blue) and labelled peptide (green) images. The staining was less intense after 60 min, but the nuclear localisation of the labelled peptide remained very clear. The confocal microscopy images are taken at the plane of the center of cell nuclei, indicating that dye associated with the nuclei is likely to be intranuclear. In live cell imaging, the labelled peptide appeared less diffuse, as indicated by the punctate red staining. In this experiment the labelled peptide was associated with cell nuclei at 40 and 60 min, and even more so after 90 min. As with the fixed cell imaging, these images are taken at the plane of the center of cell nuclei, indicating that the labelled peptide associated with nuclei is likely to be intranuclear. These experiments indicate that there is rapid nuclear uptake of peptide #66 by these breast cancer cells, consistent with the data that a 1-h preincubation of cells with peptide #66 is sufficient to inhibit the formation of complexes between IGFBP-3 and NONO/SFPQ as shown in FIG. 6.

### Example 8

### Peptide #66 Inhibits the Complex Formation Between IGFBP-3 and NONO/SFPQ in Glioblastoma Cell Lines

Glioblastoma represents another type of IGFBP-3 expressing cancer. It was demonstrated that in 2 glioblastoma cell lines, A172 and M059K, etoposide stimulated the formation of complexes between IGFBP-3 and NONO/SFPQ as seen in breast cancer cells (FIG. 9a). The cell line, M059K, was used to test the effects of peptide #66 on these complexes (FIG. 9b). The human glioblastoma cell line A172 was maintained in DMEM with 10% fetal calf serum, and M059K cells were maintained in DMEM/F12 containing 10% fetal calf serum under standard conditions. A172 or M059K human glioblastoma cells were exposed to 20 μM of etoposide (Etop) for the indicated times (0, 1, 2, or 4 h), after which NONO and SFPQ were precipitated from cell lysates by anti-IGFBP-3 antiserum (Fab fraction) coupled to agarose beads. The samples were blotted for NONO or SFPQ after fractionation by SDS-PAGE. For the experiment shown in FIG. 9b, M059K human glioblastoma cells were incubated for 1 h without (control) or with 25 μM of peptide #66 prior to exposure to 20 μM of etoposide (Etop) for the indicated times (0, 1, 2, or 4 h), after which NONO and SFPQ were precipitated from cell lysates by anti-IGFBP-3 antiserum (Fab fraction) coupled to agarose beads. The samples were blotted for NONO or SFPQ after fractionation by SDS-PAGE. FIG. 9a demonstrates that human glioblastoma cell lines form the same etoposide-stimulated complexes containing IGFBP-3 and NONO/SFPQ as previously observed to form in breast cancer cell lines. FIG. 9b shows that the formation of these complexes is inhibited by exposure of the cells to peptide #66, suggesting that the peptide is likely to inhibit DNA damage repair and therefore have an effect on glioblastoma cell survival as observed for breast cancer cells.

1. An agent that inhibits the interaction between IGFBP-3 and NONO.

2. An isolated peptide comprising residues:
X₁-X₂-X₃-X₄-X₅-X₆-X₇-X₈-X₉,
wherein X₁ is His, X₂ is Leu, X₃ is Lys, X₄ is Phe, X₅ is Leu, X₆ is Asn, X₇ is Val, X₈ is Leu and X₉ is Ser, or conservative substitutions thereof,
or a pharmaceutically acceptable salt of the peptide.

3. The peptide of claim 2, wherein the peptide comprises the sequence:
His-Leu-Lys-Phe-Leu-Asn-Val-Leu-Ser-Pro-Arg-Gly,
or conservative substitutions thereof,
or a pharmaceutically acceptable salt of the peptide.

4. The peptide of claim 2, wherein the peptide comprises the sequence:
Thr-Leu-Asn-His-Leu-Lys-Phe-Leu-Asn-Val-Leu-Ser,
or conservative substitutions thereof,
or a pharmaceutically acceptable salt thereof.

5. (canceled)

6. (canceled)

7. (canceled)

8. A pharmaceutical composition comprising an agent of claim 1, and optionally at least one pharmaceutically acceptable excipient.

9. The pharmaceutical composition of claim 8 further comprising a chemotherapeutic agent, a radiomimetic agent or a PARP inhibitor.

10. The pharmaceutical composition of claim 9, wherein the chemotherapeutic agent is selected from the group consisting of a bifunctional alkylator, a monofunctional alkylator, a topoisomerase inhibitor, an antimetabolite, a replication inhibitor and a platinum drug.

11. The pharmaceutical composition of claim 10, wherein the chemotherapeutic agent is etoposide.

12. The pharmaceutical composition of claim 9, wherein the PARP inhibitor is selected from the group consisting of veliparib and olaparib.

13. A pharmaceutical composition comprising an isolated peptide of claim 2, and optionally at least one pharmaceutically acceptable excipient.

14. The pharmaceutical composition of claim 13 further comprising a chemotherapeutic agent, a radiomimetic agent or a PARP inhibitor.

15. The pharmaceutical composition of claim 14, wherein the chemotherapeutic agent is selected from the group consisting of a bifunctional alkylator, a monofunctional alkylator, a topoisomerase inhibitor, an antimetabolite, a replication inhibitor and a platinum drug.

16. The pharmaceutical composition of claim 15, wherein the chemotherapeutic agent is etoposide.

17. The pharmaceutical composition of claim 14, wherein the PARP inhibitor is selected from the group consisting of veliparib and olaparib.

18. A method of enhancing chemosensitivity or radiosensitivity in cancer treatment comprising administering to a subject in need thereof a therapeutically effective amount of an agent of claim 1, wherein the cancer is an IGFBP-3 expressing cancer.

19. The method of claim 18, wherein the IGFBP-3 expressing cancer is breast cancer, prostate cancer, pancreatic cancer, glioblastoma cancer or Triple Negative Breast Cancer (TNBC).

20. A method of enhancing chemosensitivity or radiosensitivity in cancer treatment comprising administering to a subject in need thereof a therapeutically effective amount of an isolated peptide of claim 2, wherein the cancer is an IGFBP-3 expressing cancer.

21. The method of claim 20, wherein the IGFBP-3 expressing cancer is breast cancer, prostate cancer, pancreatic cancer, glioblastoma cancer or Triple Negative Breast Cancer (TNBC).

22. A method of enhancing chemosensitivity or radiosensitivity in TNBC treatment comprising administering to a subject in need thereof a therapeutically effective amount of an agent of claim 1.

23. A method of enhancing chemosensitivity or radiosensitivity in TNBC treatment comprising administering to a subject in need thereof an isolated peptide of claim 2.
